(19) United States
(12) Patent Application Publication
(10) Pub. No.: US 2020/0385489 A1
(43) Pub. Date: Dec. 10, 2020

(54) USING COLIPASE INHIBITORS TO TREAT PANCREATITIS

(71) Applicant: Mayo Foundation for Medical Education and Research, Rochester, MN (US)

(72) Inventor: Vijay P. Singh, Scottsdale, AZ (US)

(21) Appl. No.: 17/002,665

(22) Filed: Aug. 25, 2020

Related U.S. Application Data
(63) Continuation of application No. 15/773,450, filed on May 3, 2018.
(60) Provisional application No. 62/250,375, filed on Nov. 3, 2015.

(51) Int. Cl. C07K 16/40; C07K 16/18; A61K 31/17; A61P 1/18; A61K 31/365

(52) U.S. Cl. CPC C07K 16/40; C07K 16/18; A61K 2039/505; A61P 1/18; A61K 31/365; A61K 31/17

(57) ABSTRACT

This document provides materials and methods for treating pancreatitis (e.g., severe acute pancreatitis), a complication associated with pancreatitis (e.g., organ failure), and/or an acute inflammatory condition in a mammal (e.g., inflammation from a burn or trauma). For example, methods and materials for using one or more colipase inhibitors to treat a mammal having pancreatitis (e.g., acute pancreatitis) are provided.

### CROSS-REFERENCE TO RELATED APPLICATIONS

This application is a continuation of U.S. application Ser. No. 15/773,450, filed May 3, 2018, which is a National Stage application under 35 U.S.C. § 371 of International Application No. PCT/US2016/060116, having an International Filing Date of Nov. 2, 2016, which claims priority to U.S. Application Ser. No. 62/250,375, filed on Nov. 3, 2015. This disclosure of the prior applications are considered part of (and are incorporated by reference in) the disclosure of this application.

### TECHNICAL FIELD

This document relates to materials and methods for treating pancreatitis (e.g., severe acute pancreatitis), a complication associated with pancreatitis (e.g., organ failure), and/or an acute inflammatory condition in a mammal (e.g., inflammation from a burn or trauma). For example, this document provides methods for using one or more colipase inhibitors to treat a mammal having pancreatitis (e.g., acute pancreatitis).

### BACKGROUND

Fat necrosis is specialized necrosis of fat tissue, resulting from the action of activated lipases on fatty tissues. In the pancreas, fat necrosis occurs in acute pancreatitis, a condition where the pancreatic enzymes leak out into the peritoneal cavity, liquefy the membrane, and split the triglyceride esters into fatty acids with fat saponification.

Acute pancreatitis occurs in about 30 per 100,000 people a year (Lankisch et al., Lancet (2015) 386:85-96). New cases of chronic pancreatitis develop in about 8 per 100,000 people a year and currently affect about 50 per 100,000 people in the United States (Muniraj et al., Diseaseamonth: DM (2014) 60:530-50). Globally, in 2013, pancreatitis resulted in 123,000 deaths up from 83,000 deaths in 1990 (GBD 2013 Mortality and Causes of Death, Collaborators Lancet (2014) 385:117-71). Severe acute pancreatitis has mortality rates around 29%, which may be higher when necrosis of the pancreas has occurred (Munoz et al., Am Fam Physician (2000) 62:164-74). Acute pancreatitis is usually treated with intravenous fluids, pain medication, and sometimes antibiotics.

### SUMMARY

This document provides materials and methods for treating pancreatitis (e.g., severe acute pancreatitis), a complication associated with pancreatitis (e.g., organ failure), and/or an acute inflammatory condition in a mammal (e.g., inflammation from a burn or trauma). For example, this document provides methods and materials for administering one or more colipase inhibitors to a mammal having pancreatitis (e.g., acute pancreatitis) under conditions wherein the severity of pancreatitis is reduced. A colipase inhibitors can be an inhibitor of colipase polypeptide expression or an inhibitor of colipase polypeptide activity.

As demonstrated herein, colipase inhibitors can be effective to treat pancreatitis, a complication associated with pancreatitis, and/or an acute inflammatory condition. In some cases, one or more colipase inhibitors can be used to reduce the susceptibility of developing pancreatitis, a complication associated with pancreatitis, and/or an acute inflammatory condition. In some cases, one or more colipase inhibitors can be used to reduce or slow the progression of pancreatitis, a complication associated with pancreatitis, and/or an acute inflammatory condition.

In general, one aspect of this document features a method for treating pancreatitis in a mammal. The method comprises, or consist essentially of, (a) identifying the mammal as having pancreatitis, and (b) administering an inhibitor of colipase polypeptide activity to the mammal. The mammal can be a human. The inhibitor can be orlistat or an anti-colipase antibody. The pancreatitis can be acute pancreatitis. The acute pancreatitis can be severe acute pancreatitis. The complication associated with pancreatitis can be selected from the group consisting of shock, infection, systemic inflammatory response syndrome, organ failure, fat necrosis, and lipotoxicity. The complication associated with pancreatitis can be shock comprising visceral ischemia. The complication associated with pancreatitis can be organ failure comprising renal failure or respiratory failure. The complication associated with pancreatitis can be fat necrosis. The acute inflammatory condition can be associated with pancreatitis, burn, or trauma.

In another aspect, this document features a method for treating a complication associated with pancreatitis in a mammal. The method comprises, or consists essentially of, (a) identifying the mammal as having the complication associated with pancreatitis, and (b) administering an inhibitor of colipase polypeptide activity to the mammal. The mammal can be a human. The inhibitor can be orlistat or an anti-colipase antibody. The pancreatitis can be acute pancreatitis. The acute pancreatitis can be severe acute pancreatitis. The complication associated with pancreatitis can be selected from the group consisting of shock, infection, systemic inflammatory response syndrome, organ failure, fat necrosis, and lipotoxicity. The complication associated with pancreatitis can be shock comprises visceral ischemia. The complication associated with pancreatitis can be organ failure comprising renal failure or respiratory failure. The complication associated with pancreatitis can be fat necrosis. The acute inflammatory condition can be associated with pancreatitis, burn, or trauma.

In another aspect, this document features a method for treating an acute inflammatory condition in a mammal. The method comprises, or consists essentially of, (a) identifying the mammal as having the acute inflammatory condition, and (b) administering an inhibitor of colipase polypeptide activity to the mammal. The mammal can be a human. The inhibitor can be orlistat or an anti-colipase antibody. The pancreatitis can be acute pancreatitis. The acute pancreatitis can be severe acute pancreatitis. The complication associated with pancreatitis can be selected from the group consisting of shock, infection, systemic inflammatory response syndrome, organ failure, fat necrosis, and lipotoxicity. The complication associated with pancreatitis can be shock comprises visceral ischemia. The complication associated with pancreatitis can be organ failure comprising renal failure or respiratory failure. The complication associated with pancreatitis can be fat necrosis. The acute inflammatory condition can be associated with pancreatitis, burn, or trauma.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1(A-D) show colipase is present in fat necrosis during caerulein pancreatitis. FIG. 1A shows fat pads of untreated, control obese (Ob/Ob) mice. FIG. 1B shows fat necrosis (FN) in the fat pads of obese (Ob/Ob) mice having caerulein pancreatitis (CR). FIG. 1C shows a western blot of the fat pads which revealed colipase (CLPS), pancreatic lipase related protein-2 (PLRP2), pancreatic triglyceride lipase (PTL), and bile salt dependent lipase (BSDL) to be present in the FN but absent in the control fat pads, and revealed adipocyte triglyceride lipase (ATGL) and hormone sensitive lipase (HSL) to be present in the control fat pads but absent in the FN. Na/K ATPase served as a loading control. FIG. 1D shows an increase in pancreatic lipase activity in the fat pads of the mice with caerulein pancreatitis (CR).

FIGS. 2(A-B) show colipase is essential to lipolysis of adipocyte triglyceride by pancreatic lipases. FIG. 2A shows lactase dehydrogenase (LDH) leakage into the medium from primary mouse adipocytes incubated with mouse PLRP2 (1 microgram/mL) alone or with colipase. FIG. 2B shows glycerol release from primary mouse adipocytes incubated with mouse PLRP2 alone or with colipase.

FIGS. 3(A-B) show colipase increases fat necrosis in cells induced by pancreatic triglyceride lipase (PTL). FIG. 3A shows PTL alone resulted in a 5-8% lactase dehydrogenase (LDH) leakage compared to controls, colipase increased LDH leakage to 30-40%. LDH leakage was prevented by the lipase inhibitor orlistat (20 micromolar). FIG. 3B shows glycerol release from primary mouse adipocytes incubated with PTL alone or with colipase, colipase increased glycerol release. Glycerol release was prevented by the lipase inhibitor orlistat. Con=control.

FIG. 4 shows fatty acid release from cells after transfection of pancreatic triglyceride lipase (PTL) with or without colipase. Fatty acid release only occurred in the presence of colipase.

FIG. 5 shows fatty acid release from cells after transfection of pancreatic lipase related protein-2 (PLRP2) with or without lipase. Fatty acid release only occurred in the presence of colipase.

FIGS. 6 (A-B) show colipase increases cytokine release induced by pancreatic lipases. FIG. 6A shows IL-6 release into the medium when both colipase and PTL were present. Cytokine release was prevented by orlistat. FIG. 6B shows MCP-1 release into the medium when both colipase and PTL were present. Con=control.

FIG. 7 shows flow cytometry of peripheral blood mononuclear cells (PBMCs). Exposure of PBMCs to the medium of PLRP2+colipase treated adipocytes causes PBMC cell death.

FIG. 8 shows an increase in apoptotic cells in the circulation of rats with lethal pancreatitis (CER+GTO), which is prevented by lipase inhibition with orlistat (Orli). Con=control; CER=cerulein pancreatitis; GTO=glyceryl trioleate.

FIG. 9 shows that colipase neutralization can prevent the lipotoxic cell death induced by pancreatic lipases during pancreatitis. Lactase dehydrogenase (LDH) leakage was prevented by colipase antibody (Ab) and by inhibiting lipolysis with orlistat (Orli). LDH leakage was not prevented by the unimmunized serum (Con Sr). G=triglyceride glyceryl tri-linoleate; hP=human pancreatic triglyceride lipase; C=human colipase.

FIGS. 10 (A-C) show that pancreatic lipases mediate fat necrosis dependent acinar necrosis. FIG. 10A shows that atglistatin, but not orlistat, can prevent isoproterenol (Iso) induced lipolysis in adipocytes (Adipo). FIG. 10B shows acini. FIG. 10C shows that orlistat, but not atglistatin, inhibited adipocyte (Adipo) induced necrosis, measured by propidium iodide (PI) uptake, in adipocyte co-cultures.

### DETAILED DESCRIPTION

This document provides methods and materials for treating pancreatitis. For example, this document provides methods and materials for using colipase inhibitors to treat pancreatitis (e.g., severe acute pancreatitis), a complication associated with pancreatitis (e.g., organ failure), and/or an acute inflammatory condition in a mammal (e.g., inflammation from a burn or trauma). In some cases, one or more colipase inhibitors can be used to reduce the susceptibility of developing pancreatitis, a complication associated with pancreatitis, and/or an acute inflammatory condition. In some cases, one or more colipase inhibitors can be used to reduce or slow the progression of pancreatitis, a complication associated with pancreatitis, and/or an acute inflammatory condition.

When treating or reducing the risk of developing pancreatitis as described herein, the pancreatitis can be acute pancreatitis or chronic pancreatitis. Acute pancreatitis can be caused by, for example, gallstones, heavy alcohol use, direct trauma, certain medications (e.g., corticosteroids, thiopurines, or neucleoside reverse transcriptase inhibitors), infections (e.g., mumps or coxsackie virus infections), or tumors. Acute pancreatitis can be mild acute pancreatitis (causing no complications) or severe acute pancreatitis (causing complications). Chronic pancreatitis is commonly due heavy alcohol use and can develop from acute pancreatitis. In some embodiments, the pancreatitis treated as described herein can be acute pancreatitis. In some embodiments, the pancreatitis treated as described herein can be severe acute pancreatitis.

In some cases, the materials and methods provided herein can be used to treat a complication associated with pancreatitis. A complication associated with pancreatitis can include, without limitation, shock (e.g., hypovolemic, circulatory, and/or septic), infection, systemic inflammatory response syndrome, organ failure (e.g., renal failure and/or respiratory failure), fat necrosis, and apoptosis (e.g., peripheral blood mononuclear cell (PBMC) cell death and/or lipotoxic cell death). Additional examples of complications associated with pancreatitis can include, without limitation, recurrent pancreatitis, pancreatic pseudocysts, and pancreatic abscess.

In some cases, the materials and methods provided herein can be used to treat an acute inflammatory condition. Examples of acute inflammatory conditions that can be treated as described herein include, without limitation, inflammation associated with pancreatitis, burn, or trauma.

Any type of mammal having pancreatitis (or a complication associated with pancreatitis and/or an acute inflammatory condition) or at risk for developing pancreatitis (or a complication associated with pancreatitis and/or an acute inflammatory condition) can be treated as described herein. For example, humans and other primates such as monkeys having pancreatitis can be treated with one or more colipase inhibitors. In some cases, dogs, cats, horses, cows, pigs, sheep, rabbits, mice, and rats can be treated with one or more colipase inhibitors as described herein.

Any appropriate method can be used to identify a mammal having pancreatitis (or a complication associated with pancreatitis and/or an acute inflammatory condition) or as being at risk for developing pancreatitis (or a complication associated with pancreatitis and/or an acute inflammatory condition). For example, abdominal ultrasound, computerized tomography (CT) scan, and blood tests (e.g., for an increase in amylase and/or lipase such as a level greater than about threefold the upper limit of normal) can be used to identify a human or other mammal having pancreatitis.

Once identified as having pancreatitis (or a complication associated with pancreatitis and/or an acute inflammatory condition) or as being at risk for developing pancreatitis (or a complication associated with pancreatitis and/or an acute inflammatory condition), the mammal can be administered or instructed to self-administer one or more colipase inhibitors (e.g., a composition containing one or more colipase inhibitors that reduce colipase polypeptide expression and/or activity). A colipase inhibitors can be an inhibitor of colipase polypeptide expression or an inhibitor of colipase polypeptide activity. Examples of compounds that reduce colipase polypeptide expression include, without limitation, nucleic acid molecules designed to induce RNA interference (e.g., an RNAi molecule or a shRNA molecule), antisense molecules, and miRNAs. Examples of inhibitors of colipase polypeptide activity include, without limitation, orlistat and anti-colipase antibodies.

In some cases, one or more colipase inhibitors (e.g., one, two, three, four, five, or more colipase inhibitors) can be administered to a mammal to treat pancreatitis (e.g., to reverse pancreatitis), a complication associated with pancreatitis (e.g., organ failure), and/or an acute inflammatory condition in a mammal (e.g., inflammation from a burn or trauma). For example, two or more colipase inhibitors can be administered to a mammal (e.g., a human with pancreatitis) to treat pancreatitis (e.g., to reverse pancreatitis). In some cases, one or more colipase inhibitors can be formulated into a pharmaceutically acceptable composition for administration to a mammal having pancreatitis or being at risk for developing pancreatitis. For example, a therapeutically effective amount of a colipase inhibitor can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more colipase inhibitors can be designed for oral or parenteral (including subcutaneous, intramuscular, intravenous, intraperitoneal, and intradermal) administration. When being administered orally, a pharmaceutical composition containing one or more colipase inhibitors can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Such injection solutions can be in the form, for example, of a sterile injectable aqueous or oleaginous suspension. This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1,3-butanediol. Examples of acceptable vehicles and solvents that can be used include, without limitation, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a pharmaceutically acceptable composition including one or more colipase inhibitors can be administered locally or systemically. For example, a composition containing a colipase inhibitor can be administered systemically by an oral administration or by injection to a mammal (e.g., a human).

Effective doses can vary depending on the severity of the pancreatitis, the route of administration, the age and general health condition of the subject, excipient usage, the possibility of co-usage with other therapeutic treatments such as use of other agents, and the judgment of the treating physician.

An effective amount of a composition containing one or more colipase inhibitors can be any amount that reduces the severity of a symptom of a condition being treated (e.g., pancreatitis) without producing significant toxicity to the mammal. For example, an effective amount of a colipase inhibitor such as orlistat can be from about 0.01 mg/kg to about 50 mg/kg (e.g., from about 0.1 mg/kg to about 50 mg/kg, from about 1 mg/kg to about 50 mg/kg, from about 5 mg/kg to about 50 mg/kg, from about 10 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg/kg to about 1 mg/kg, from about 0.1 mg/kg to about 10 mg/kg, or from about 1 mg/kg to about 5 mg/kg). In some cases, between about 3 g twice-weekly of a colipase inhibitor can be administered to an average sized human (e.g., about 65-75 kg human) daily for about four to about eight weeks (e.g., about five to six weeks). If a particular mammal fails to respond to a particular amount, then the amount of a colipase inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., pancreatitis) may require an increase or decrease in the actual effective amount administered.

The frequency of administration can be any frequency that reduces the severity of a symptom of a condition to be treated (e.g., pancreatitis) without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing one or more colipase inhibitors can include rest periods. For example, a composition containing one or more colipase inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., pancreatitis) may require an increase or decrease in administration frequency. An effective duration for administering a composition containing one or more colipase inhibitors can be any duration that reduces the severity of a symptom of the condition to be treated (e.g., pancreatitis) without producing significant toxicity to the mammal. For example, the effective duration can vary from several days to several weeks, months, or years. In some cases, the effective duration for the treatment of pancreatitis can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated (e.g., pancreatitis) can be monitored. Any appropriate method can be used to determine whether or not the severity of a symptom is reduced. For example, the severity of a symptom of pancreatitis (e.g., fat necrosis and/or lipotoxicity) can be assessed using abdominal ultrasound, CT scan, and/or blood tests (e.g., for an increase in amylase and/or lipase) at different time points.

In some cases, one or more colipase inhibitors (e.g., one, two, three, four, five, or more colipase inhibitors) can be used in combination with another pancreatitis treatment. For example, administration of one or more colipase inhibitors can be used on combination with endoscopic retrograde cholangiopancreatography, surgical removal of the gallbladder, surgical removal of pancreatic fluid, cessation or reduction of alcohol consumption, administration of pain medicine, and/or administration of pancreatic enzyme supplements.

The invention will be further described in the following examples, which do not limit the scope of the invention described in the claims.

### EXAMPLES

### Example 1: Colipase is Present in Fat Necrosis During Caerulein Pancreatitis

Pancreatitis was induced in obese (Ob/Ob) mice. Ob/Ob mice (50-60 gm) were either given caerulein (50 mcg/kg/hour) to induce pancreatitis or left untreated as control animals.

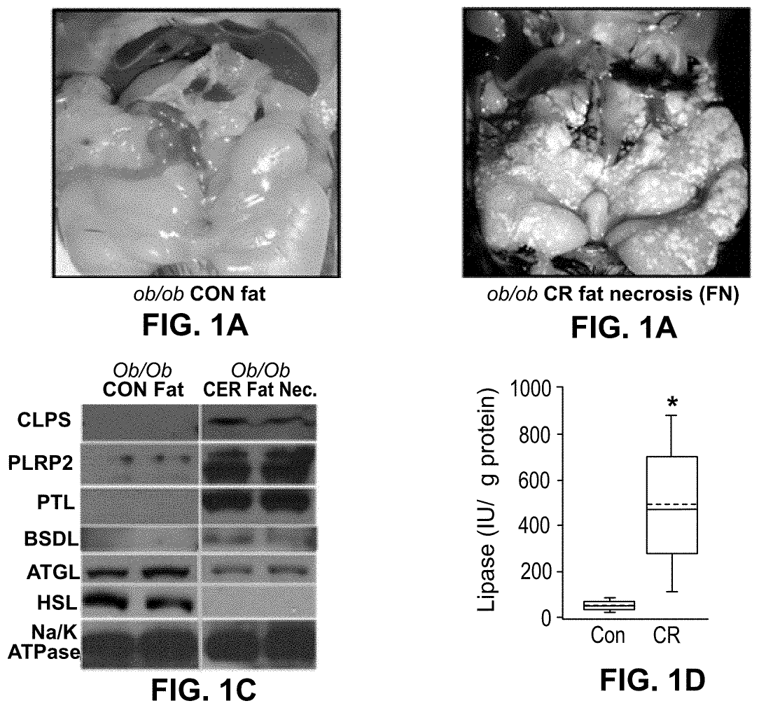

Caerulein treatment resulted in mortality (100% in 3 days) with severe pancreatitis, fat necrosis and saponification of the hydrolyzed fat seen as cheesy white round opacities in the fat pads (FIG. 1B).

Western blot analysis of the fat pads from caerulein treated mice revealed colipase to be present in the fat necrosis (FIG. 1C). Also present in the fat pads from caerulein treated mice were pancreatic lipase related protein-2 (PLRP2), pancreatic triglyceride lipase (PTL), and bile salt dependent lipase (BSDL), which were absent in the control fat pads (FIG. 1C). Western blot analysis also revealed that adipocyte triglyceride lipase (ATGL) and hormone sensitive lipase (HSL) to be present in the control fat pads but absent in the FN (FIG. 1C).

The increased amounts of pancreatic lipases and colipase was also associated with an increase in pancreatic lipase activity in the fat pads of the mice with caerulein pancreatitis (FIG. 1D).

These results show that pancreatic lipases and colipase are present in fat necrosis and the saponification consequent to hydrolysis of the adipocyte triglyceride.

### Example 2. Colipase is Essential to Lipolysis of Adipocyte Triglyceride by Pancreatic Lipases

Primary mouse adipocytes were incubated with mouse PLRP2 (1 microgram/mL) alone or with colipase (0.25 microgram/mL).

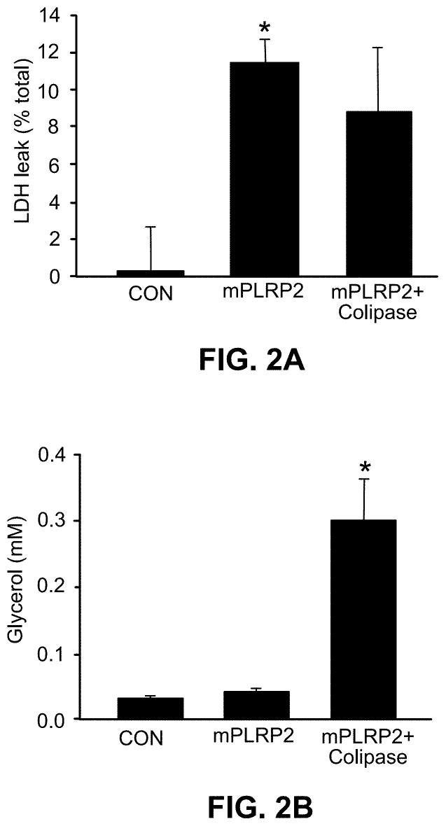

Lactase dehydrogenase (LDH) leakage into the medium was evaluated as a marker of adipocyte damage. PLRP2 alone caused adipocyte damage, resulting in LDH leakage (FIG. 2A).

Glycerol release into the medium was examined as a marker of lipolysis. Lipolysis of the adipocyte triglyceride required both colipase and PLRP2 (FIG. 2B).

These results show that colipase plays an essential role in the pancreatic lipase mediated lipolysis of fat resulting in fat necrosis.

### Example 3. Colipase Increases Fat Necrosis Induced by Pancreatic Lipases

Differentiated 3T3-LI adipocytes with lipid droplets were transfected with adenovirus expressing pancreatic triglyceride lipase (PTL) to induce lactate dehydrogenase (LDH) leakage or left as unstimulated controls. The medium was replaced with serum free medium alone or with colipase (0.25 mcg/mL). In some wells the lipase inhibitor orlistat (20 micromolar) was added every 2 hours.

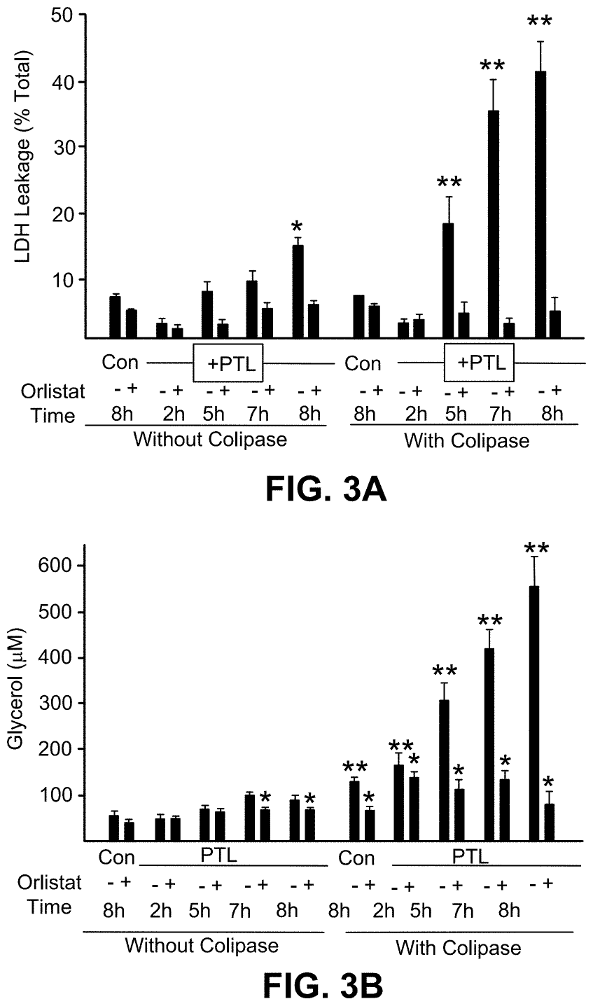

LDH leakage into the medium was measured as a marker of cell injury. PTL alone resulted in a 5-8% LDH leakage compared to controls over 8 hours. Colipase increased this to 30-40% LDH leakage compared to controls. LDH leakage was completely prevented by orlistat (FIG. 3A). Glycerol release was measure as a marker of lipolysis. PTL alone resulted in an increase glycerol release compared to controls over 8 hours. Colipase increased glycerol release compared to controls. Glycerol release was prevented by the lipase inhibitor orlistat (FIG. 3B).

These results show that lipolysis of adipocyte triglycerides by pancreatic lipases along with colipase results in adipocyte and fat necrosis.

### Example 4: Colipase Causes Release of Fatty Acids and Cytokines

Differentiated 3T3-LI adipocytes with lipid droplets were transfected with adenovirus expressing pancreatic triglyceride lipase (PTL) or left as unstimulated controls. The medium was replaced with serum free medium alone or with colipase (0.25 mcg/ml). Fatty acid release into the medium was measured at 8 and 24 hours after colipase addition.

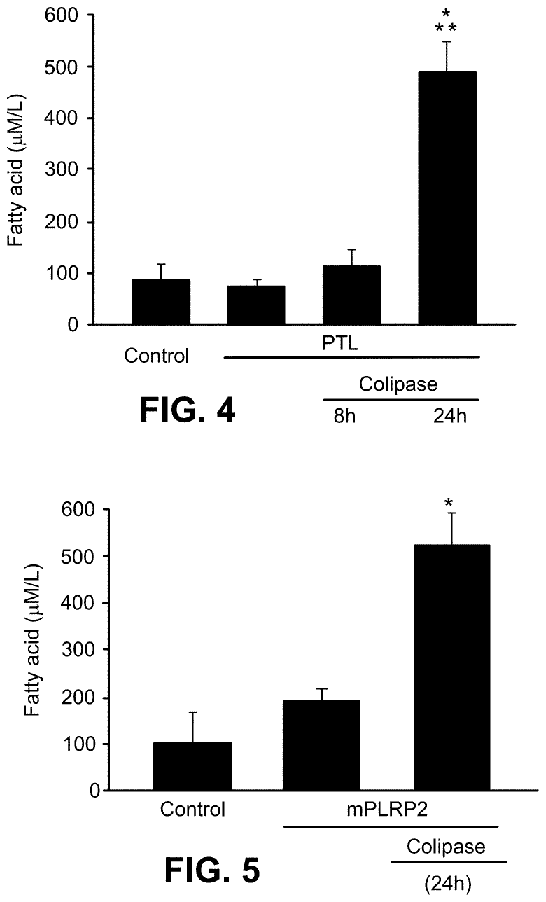

Fatty acid release only occurred in the presence of colipase (FIG. 4).

These results shows that colipase is essential for the lipolysis of adipocyte triglyceride by PTL.

### Example 5. Colipase Causes Fatty Acid Release after Transfection of Pancreatic Lipases

Differentiated 3T3-LI adipocytes with lipid droplets were transfected with adenovirus expressing pancreatic lipase related protein-2 (PLRP2) or left as unstimulated controls. The medium was replaced with serum free medium alone or with colipase (0.25 mcg/mL). Fatty acid release into the medium was measured at 24 hours after colipase addition.

Fatty acid release only occurred in the presence of colipase (FIG. 5).

These results show that colipase is essential for the lipolysis of adipocyte triglyceride by PLRP2.

### Example 6. Colipase Increases Cytokine Release Induced by Pancreatic Lipases During Fat Necrosis

Differentiated 3T3-LI adipocytes with lipid droplets were transfected with adenovirus expressing pancreatic triglyceride lipase (PTL) or left as unstimulated controls. The medium was replaced with serum free medium alone or with colipase (0.25 mcg/mL). In some wells the lipase inhibitor orlistat (20 micromolar) was added every 2 hours.

Cytokine release was measured as a marker of inflammatory response.

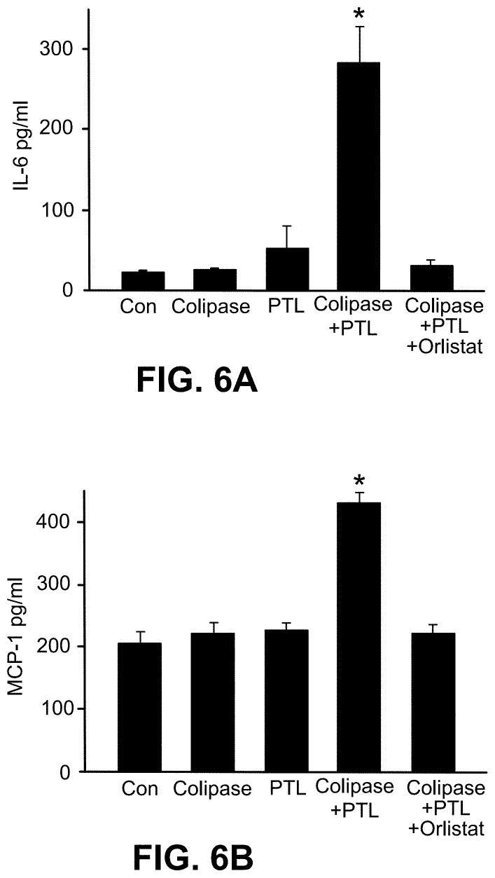

IL-6 release into the medium was measured by luminex. There was a significant release of IL-6 only when both colipase and PTL were present. This release was prevented by orlistat (FIG. 6A).

MCP-1 release into the medium was measured by luminex. There was a significant release of MCP-1 only when both colipase and PTL were present. This release was prevented by orlistat (FIG. 6B).

These results show that colipase dependent lipolysis is essential for the inflammatory response in fat necrosis.

### Example 7: Peripheral Blood Cell Apoptosis During Pancreatitis

Differentiated 3T3-LI adipocytes with lipid droplets were transfected with adenovirus expressing pancreatic lipase related protein-2 (PLRP2) or left as unstimulated controls. Colipase (0.25 mcg/mL) was added to the medium of PLRP2 treated cells. Supernatants were taken from these conditions and added to peripheral blood mononuclear cells (PBMCs). These cells were then stained with fluorescein isothiocyanate (FITC) conjugated annexin V (a marker of apoptosis) and propidium iodide (a marker of necrosis), washed, and fixed. Staining was quantified on the Fortessa flow cytometer.

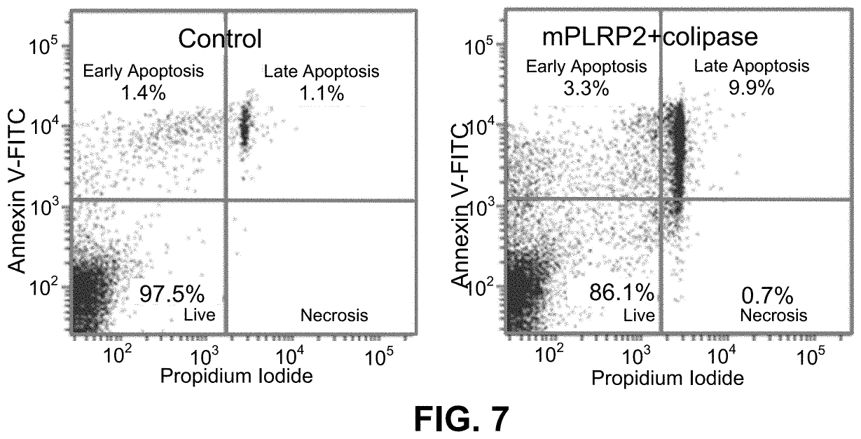

Control cells had excellent viability (97.5%; FIG. 7, left panel). Cells exposed to the supernatants of colipase+PLRP2 treated adipocytes had an 11.4% decrease in viability, with an increase in both apoptotic and necrotic PBMCs (FIG. 7, right panel).

These results show that exposure of PBMC to the medium of PLRP2+colipase treated adipocytes caused cell death.

### Example 8. Lipase Inhibition Prevents Peripheral Blood Cell Apoptosis During Pancreatitis

Pancreatitis was induced in rats and flow cytometry was performed on white blood cells (WBCs) from peripheral blood.

Rats (100 gm rats) were either given caerulein (20 mcg/kg BID (twice a day) intraperitoneally) to induce pancreatitis or left untreated as control animals. Some rats were also given glyceryl trioleate (3 ml intraperitoneally) to induce lactate dehydrogenase (LDH) leakage. Some rats also received the lipase inhibitor orlistat (50 mg/kg). Blood was collected from rats 2 days after induction of caerulein pancreatitis with or without administration of caerulein, glyceryl trioleate, and/or orlistat.

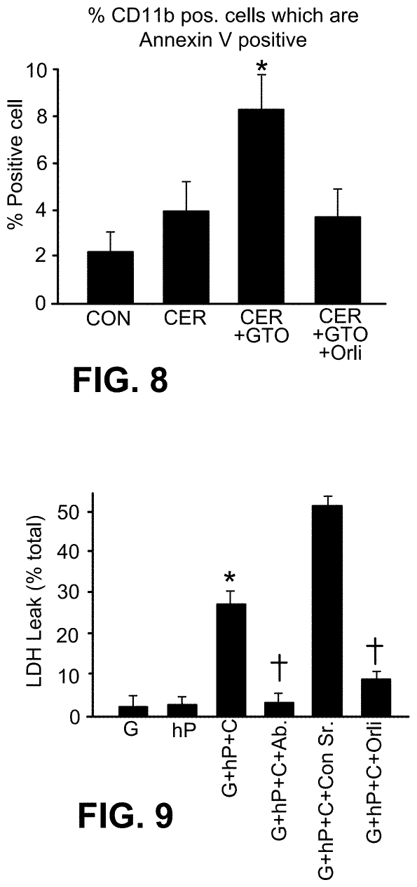

Peripheral blood cells show an increase in apoptotic CD11b positive cells in the circulation of rats with lethal pancreatitis (FIG. 8). There was 97% mortality by day 3 in the rats that received both caerulein pancreatitis and glyceryl trioleate. This was associated with a significant increase in the number of apoptotic (annexin V positive CD11b) cells. Apoptosis was prevented by orlistat. The rats that received lipase inhibition treatment survive.

These results show that colipase is involved in the death of inflammatory cells in severe acute pancreatitis and show that apoptosis is prevented by lipase inhibition with orlistat.

### Example 9: Lipase Inhibition Prevents Lactate Dehydrogenase Leakage

Rat parotid acini (which do not contain pancreatic lipases) were harvested and were exposed to glyceryl trilinoelate (300 micromolar) in combination with human pancreatic triglyceride lipase (PTL; 1 micrograms/mL) and/or human colipase (0.25 micrograms/mL). Cells exposed to glyceryl trilinoelate, PTL, and colipase were treated at a 1:4 dilution with either orlistat (50 micromolar), serum from mice immunized against human colipase, or serum from unimmunized mice. Lactose dehydrogenase (LDH) leakage into the medium was measured over 4 hours.

There was a significant increase in leakage of LDH in cells exposed to glyceryl trilinoelate, PTL, and colipase compared to controls. This increase in LDH leakage was significantly reduced by inhibition of colipase using either a colipase antibody or orlistat. LDH leakage was not affected by unimmunized serum (FIG. 9).

These results show that colipase neutralization can prevent the lipotoxic cell death induced by pancreatic lipases during pancreatitis.

### Example 10: Pancreatic Lipases Mediate Fat Necrosis Dependent Acinar Necrosis

Adipocytes were stimulated by isoproterenol (Iso 10 μM) or co-cultured with pancreatic acini. The adipose triglyceride lipase (ATGL) inhibitor atglistatin or the pancreatic lipase inhibitor orlistat (50 μM each) was added in some conditions.

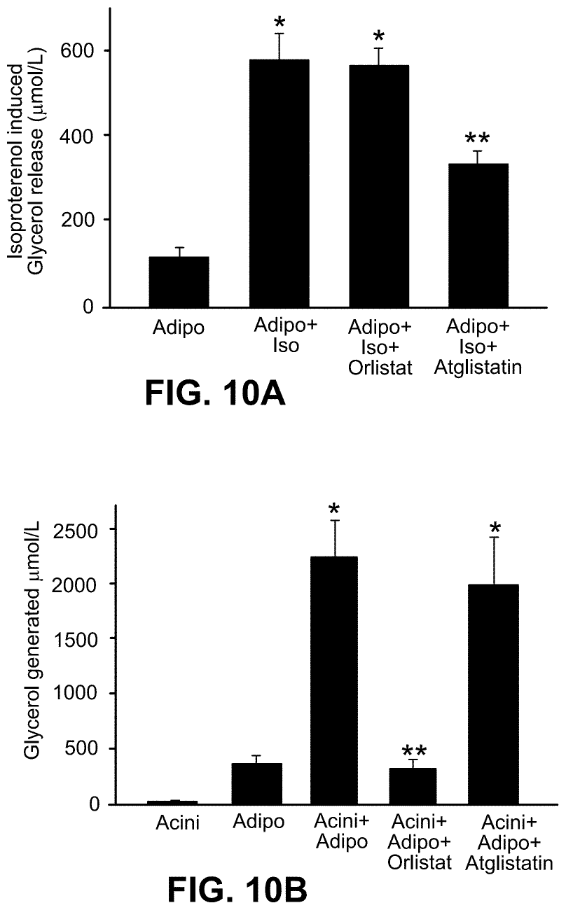

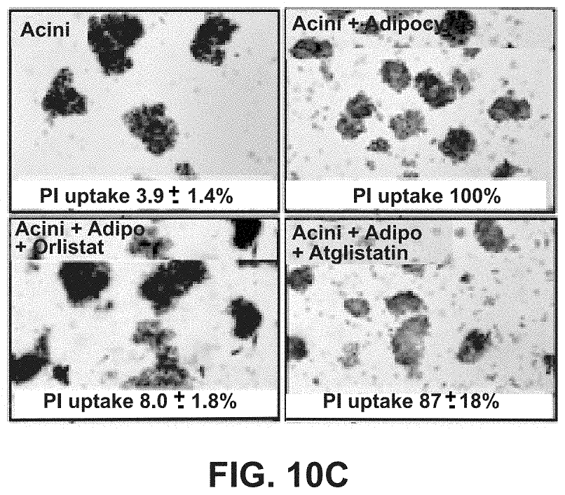

Glycerol release was measured as a marker of lipolysis in adipocytes. Isoproterenol induced lipolysis in adipocytes. Atglistatin, but not orlistat, inhibited isoproterenol induced lipolysis in adipocytes (FIG. 10A).

Co-culture of adipocytes and pancreatic acini induced necrosis in the acini. FIG. 10B shows acini. Orlistat, but not atglistatin, inhibited adipocyte induced necrosis in pancreatic acini as measured by glycerol release (FIG. 10B). Orlistat, but not atglistatin, inhibited adipocyte induced necrosis in pancreatic acini as measured by propidium iodide uptake (FIG. 10B).

These results show that while adipocyte triglyceride lipase (ATGL) mediates regulated lipolysis via isoproterenol; pancreatic lipases mediate the unregulated lipolysis during fat necrosis, causing other cells (e.g. acini) to be injured.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method for treating pancreatitis in a mammal, said method comprising:
(a) identifying said mammal as having pancreatitis, and
(b) administering an inhibitor of colipase polypeptide activity to said mammal.

2. A method for treating a complication associated with pancreatitis in a mammal, said method comprising:
(a) identifying said mammal as having said complication associated with pancreatitis, and
(b) administering an inhibitor of colipase polypeptide activity to said mammal.

3. A method for treating an acute inflammatory condition in a mammal, said method comprising:
(a) identifying said mammal as having said acute inflammatory condition, and
(b) administering an inhibitor of colipase polypeptide activity to said mammal.
